(19) United States
(12) Patent Application Publication
(10) Pub. No.: US 2024/0090636 A1
(43) Pub. Date: Mar. 21, 2024

(54) A BAG ARRANGED ON THE BODY SURFACE

(71) Applicant: Kuo Huang YANG, Taipei (TW)

(72) Inventor: Kuo Huang YANG, Taipei (TW)

(21) Appl. No.: 18/267,204

(22) PCT Filed: Dec. 17, 2021
(86) PCT No.: PCT/CN2021/139173

§ 371 Date: Jun. 14, 2023

(30) Foreign Application Priority Data
Dec. 18, 2020 (CN) 202011507300.0

(51) Int. Cl. A45C 13/10; A45C 13/36

(52) U.S. Cl. CPC A45C 13/10; A45C 13/36; A45C 13/001

(57) ABSTRACT

The present invention discloses a bag arranged on the body surface which is fixed on a body surface of a user. The bag includes a main body, a plurality of fixing parts, and a connecting part. The main body further includes a first side wall, a second side wall, a storage space, and a first opening. The fixing parts are respectively arranged on the main body, and each of the fixing parts further forms a first surface and a second surface. The second surface corresponds to the body surface of the user, and the first surface corresponds to the first side wall. The connecting part is arranged between the main body and the fixed parts to fix the main body to the fixed parts. The connecting part connects a part of the first surface and the first side wall so that the remaining part of the first surface is separated from the first side wall. In addition, the main body is positioned on the body surface of the user through the fixing parts and the connecting part.

### FIELD OF THE INVENTION

The present invention relates to the technical field of fixedly attaching bags to a body surface, especially the bag that carries foods, body fluids, or excrement.

### BACKGROUND OF THE INVENTION

Human civilization has a close relationship with containers, especially portable ones for the need of shifting around. The conventional ones are fixed by carrying and tying for the portable purpose, but the portable containers would still slide, displace or become detached. Therefore, how to improve the conventional techniques so that the portable containers would be more fixedly attached to the users is the issue to be resolved.

### SUMMARY OF THE INVENTION

The present invention provides a bag arranged on the body surface that overcomes the deficiencies of those in the prior art.

The present invention provides a bag, in an embodiment, can be easily fixed on the human body and not easily separated. Meanwhile carries materials in all kinds of states and forms, such as liquid, gas, powder, granule, and solid. For instance, the material being carried can be food, body liquids, or excrement.

In another embodiment, the present invention provides the bag as described above that includes a plurality of fixing parts being fixed to a main body by a connecting part. The connecting part fixes a first surface of each of the fixing parts on a first side wall of the main body, so as to achieve the purpose of alleviating the pulling force the bag undertakes.

In another embodiment, the present invention provides the bag as described above that has at least one of the fixing parts forming a second opening to expose the main body through a second surface thereof.

In another embodiment, the present invention provides the bag as described above that has the connecting part thereof arranged at least at one of the fixing parts, so as to form one or multiple passages.

In another embodiment, the present invention provides the bag as described above that includes a fixed member arranged through the passage(s) to fixedly (or located) attach the main body thereof to the body surface of the users.

In another embodiment, the present invention provides the bag as described above that includes reinforcement to further enhance the attachment and/or fixture of the fixing parts on at least the first surface or the second surface thereof.

In another embodiment, the present invention provides the bag as described above that has an adhesive layer arranged on at least one of the fixing parts for the main body to be fixedly attached to the body surface by the fixing part.

In another embodiment, the present invention provides the bag as described above that has the connecting part thereof arranged near the edge of the main body so that one of the fixing parts has at least parts of its edges extending out from the edge of the main body.

In another embodiment, the present invention provides the bag as described above that one of the fixing parts has said partial of the first surface thereof arranged in a distance from which to an edge of the fixing part.

In another embodiment, the present invention provides the bag as described above that the connecting part is arranged near an edge of the main body, rendering an edge of at least one of the fixing parts has at least partial thereof extending out from the edge of the main body.

In another embodiment, the present invention provides the bag as described above that includes an insulation layer arranged at least on an inner surface of the main body, an external surface of the main body, or on the first surface, so as to prevent areas such as between the first side wall and a second side wall of the main body or between the first side wall and the fixing parts from being bonded by the heat during the manufacturing process.

In another embodiment, the present invention provides the bag as described above that includes an extended element to connect a storage space of the main body to outside the main body.

In order to achieve the functions and purposes described above, the present invention provides a bag arranged on the body surface of a user. The bag comprises a main body, a plurality of fixing parts, and a connecting part. The main body includes a first side wall, a second side wall, a storage space, and a first opening. The first side wall is bonded with the second side wall, forming the storage space. The first opening is formed at least at the first side wall, at the second side wall, or in-between the first and second side walls. The storage space connects to outside the main body through the first opening. The plurality of fixing parts is individually arranged on the main body, each including a first surface and a second surface which are correspondingly arranged for the second surface to be attachable on a body surface and the first surface to be attachable on the first side wall. The connecting part is arranged in-between the main body and the fixing parts, so as to fix the fixing parts on the main body, wherein the connecting part connects partial of the first surface and the first side wall, rendering the rest of the first surface can be separated from the first side wall. Wherein the main body is attachable by the plurality of fixing parts as well as the connecting part and thereby fixed on a body surface.

In order to achieve the functions and purposes described above, the present invention provides a bag arranged on the body surface of a user. The bag comprises a main body, a plurality of fixing parts, a connecting part, and an adhesive layer. The main body includes a first side wall, a second side wall, a storage space, and a first opening. The first side wall is bonded with the second side wall, forming the storage space. The first opening is formed at least at the first side wall, at the second side wall, or in-between the first and second side walls. The storage space connects to outside the main body through the first opening. The plurality of fixing parts is individually arranged on the main body, each including a first surface and a second surface which are correspondingly arranged for the second surface to be attachable on a body surface and the first surface to be attachable on the first side wall. The connecting part is arranged in-between the main body and the fixing parts, so as to fix the fixing parts on the main body, wherein the connecting part connects partial of the first surface and the first side wall, rendering the rest of the first surface can be separated from the first side wall. The adhesive layer is formed on the second surface. Wherein the main body is attachable with the plurality of fixing parts, the connecting part, and the adhesive layer and thereby fixed on a body surface.

### BRIEF DESCRIPTION OF THE DRAWINGS

FIG. 1 is a schematic diagram illustrating the present invention in a first embodiment thereof;

FIGS. 2A and 2B are schematic diagrams illustrating the present invention in a second embodiment thereof and

FIGS. 3A and 3D are schematic diagrams illustrating the present invention in a third embodiment thereof.

### DETAILED DESCRIPTION OF THE PREFERRED EMBODIMENTS

The following describes specific embodiments of the present invention regarding the drawings, wherein the same reference numerals represent the same or similar components.

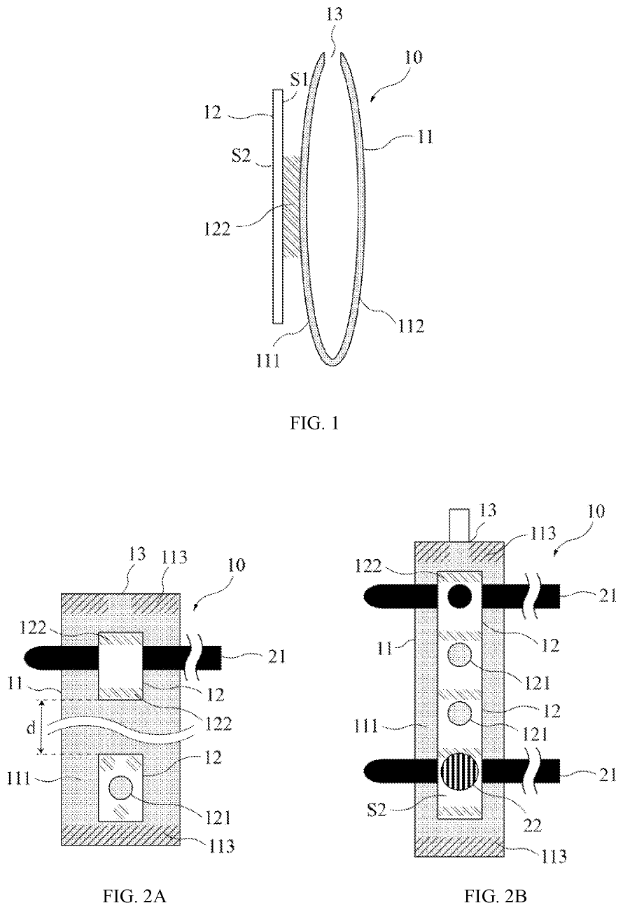

FIG. 1 is a schematic diagram illustrates the present invention in the first embodiment. In FIG. 1, the bag 10 for attaching to the body surface (not shown) includes a main body 11, a fixing part 12, and a first opening 13.

The main body 11 includes a first side wall 111 and a second side wall 112. The first side wall 111 is bonded with the second side wall 112 to form a storage space for filling in materials in liquid, gas, powders, granules, or solid states or forms. The main body 11 has its inner space connecting to outside the main body via the first opening 13. The first opening 13 can be further installed with a tube, a connector, a cover, or the adhesive layer surrounding it. Such tubes, connectors, and covers can be categorized as an extended element. For instance, when the extended element is a connector, it can be installed at the first opening 13, leaving partial of the connector exposed outside the main body 11 while another partial thereof is connected with the first opening 13.

The fixing part 12 is sheet material and includes a first surface S1 and a second surface S2 which are correspondingly arranged. Herein there is one fixing part 12 for exemplary purpose; in other embodiments, the number of the fixing parts 12 is not limited to 1. In addition, partial of the first surface S1 is connected to an external surface of the first side wall 111 of the main body 11 by a connecting part 122, rendering the rest of the first surface S1 and the external surface of the main body 11 is detached. With the fixing part 12, the users can fixedly attach the main body 11 on their body surface.

The first opening 13 carries liquids or solid materials. Herein, the first opening 13 is formed on the edge of the main body 11, an open portion where the first side wall 111 and the second side wall 112 are not bonded. Furthermore, in other embodiments, the first opening 13 can also be formed at the first side wall 111, the second side wall 112, or both

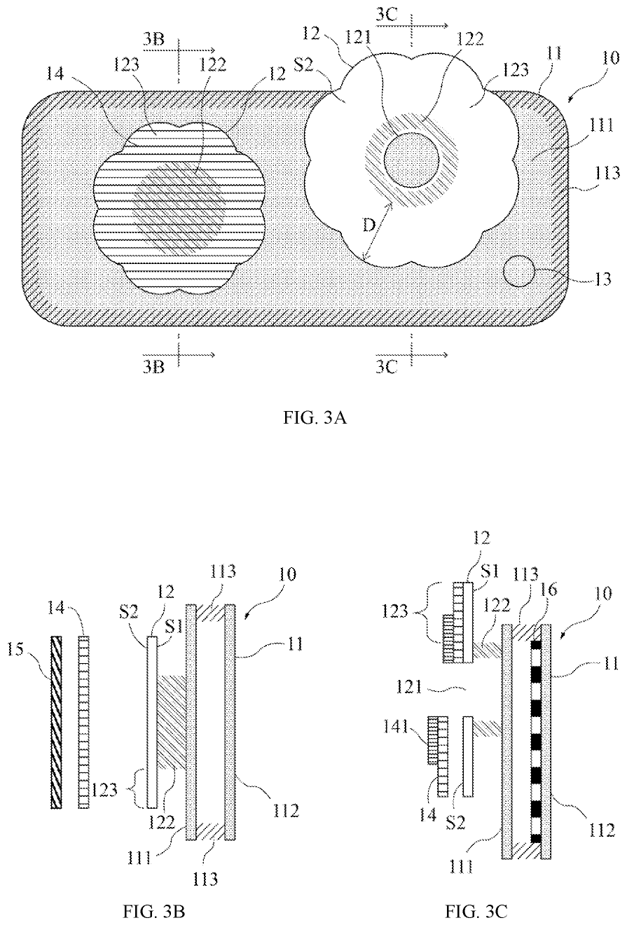

FIGS. 2A-2B are the schematic diagrams of the present invention in the second embodiment. As shown in FIG. 2A, the main body 11 is formed by having one single sheet of continuous material and bonding the left and right thereof in a folding pattern and combined with its open upper and lower edges, forming a bonded section 113 at the edge of the main body 11. Herein, the bonded section 113 is formed on the edge of the main body 11. In addition, herein the fixing parts 12 are 2 for exemplary purpose. The fixing parts 12 form a passage by two connecting parts 122; that is, the passage is formed in the space between the connecting parts 122 for arranging an external fixed member 21 such as straps and ropes therethrough. In other words, the fixing parts 12 fixedly attach the main body 11 to the body surface of the users by having the fixed member 21 passing through the passage. For the users to be able to decide where to attach it, the main body 11 can further include a plurality of fixing parts 12. This not only increases the stability of the main body 11 which will not swing or dislocate on the body surface with at least two fixing points, it also forms at least two supporting points so that the main body 11 has the gravity evenly distributed by the multiple fixing parts 12 when it is taking up certain weight. Also, the users can make their own choices upon where to pass through the straps (or ropes) with the different fixing parts 12, so as to find the best way to fix the bag 10. That is, the multiple fixing parts 12 can be arranged depending on different situations. Herein, the two fixing parts 12 have an interval d arranged in-between and they are arranged in the same direction (along the Y axis of the Figures); the interval d is spaced greater than 0 cm. In other embodiments, the interval d can also be spaced at 0 cm as shown in FIG. 2B. Besides, the fixing parts 12 are arranged in the same direction as shown in FIG. 2A, but in other embodiments, they can also be arranged in a staggered position or a combination of the same direction and the staggered position.

In another embodiment, the fixing parts 12 can be further manufactured to form a second opening 121.

Referring to FIG. 2B, the first opening 13 is engaged to a tube for exemplary purpose. The bag 10 functions as a thigh bag for storing urine. With the main body 11 disposed in a generally parallel direction with the thigh, there are four fixing parts 12 arranged vertically for exemplary purpose. Herein, the bag 10 can further include a reinforcement 22 that is arranged on the second surface S2 of the fixing parts 12. (In other embodiment, the reinforcement 22 can also be arranged on the first surface S1.) The reinforcement 22 is combined with the fixed member 21 (like straps or ropes) through the second opening 121 by hooking, engaging, adhering, or friction, in order to prevent the main body 11 from sliding or moving along the direction of the fixed member 21. On the other hand, the reinforcement 22 allows the users to easier detach the fixed member 21 from the main body 11. The reinforcement 22 can be but is not limited to hook- and loop fasteners, buttons, rivets, and adhesives. Herein, the fixed member 21 is a hook-and-loop fastener to be attached to the strap as the fixed member 21. The fixed member 21 has a size slightly larger than the second opening 121; by having the hook-and-loop fasteners of the fixed member 21 and the strap, they are tightly attached through the second opening 121 and therefore holding a steady structure of the present invention.

The connecting part 122 fixes the fixing parts 12 on the main body 11 by way of, for example, welding or adhering. For instance, as shown in FIG. 2A, when the fixing parts 12 are rectangles, the corresponding sides thereof can be attached to the main body 11 for combining; or having more than two connecting parts 122 attached to the main body 11 within the area of the rectangles is applicable. Or, as shown in FIG. 2B, the fixing parts 12 can further include the second openings 121, each forming correspondingly to one passage. What's worth mentioning is that the attaching and fixing in this embodiment is especially suitable for fixedly attaching the bag 10 on the four limbs and the torso.

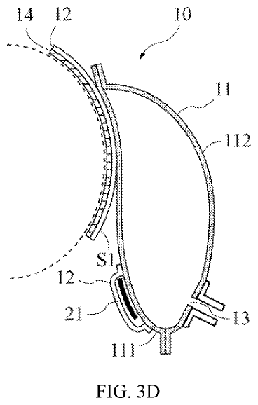

FIGS. 3A-3D are schematic diagrams of the present invention in the third embodiment. FIG. 3B is a sectional view along 3B-3B in FIG. 3A, showing the left section of the bag 10, and FIG. 3C is a sectional view along 3C-3C in FIG. 3A, showing the right section of the bag 10. FIG. 3D is a practical application view of the present invention.

Herein, the main body 11 is formed by having two pieces of sheet materials bonded at the edges thereof; the two pieces can be the same or different materials bonded by sealing. The main body 11 includes two fixing parts 12 and an adhesive layer 14 formed on the second surface S2 of the fixing parts 12 for attachment on the body surface of the users. The adhesive layer 14 can be made of, for example, pressure-sensitive adhesives that are applied on the fixing parts 12 during the manufacturing process. It can also be a double-sided tape that has one side thereof attached to the fixing parts 12 while the other side thereof attached to the users; in that case, the main body 11 would only need to replace the double-sided tape for reuse. The attaching and fixing in this embodiment is suitable on larger body surface such as the abdomen, the back, and other areas of the human body where it is not smoothly level and not easy to fixedly attach thereon. As shown in FIG. 3A, the main body 11 is in the shape of a rounded tetragon that has two pieces of thermoplastic films bonded along the edges thereof by the heat-sealing process. The bonded section 113 is the surrounding edge of the main body 11. Herein, the fixing parts 12 are made from thermoplastic films into a petal shape. Referring to FIG. 3B, the fixing parts 12 are arranged within the area between the upper and lower edges of the main body 11 and would not extend out from the inner edge of the bonded section 113 of the main body 11. In the manufacturing process, the first step is to heat seal the fixing parts 12 to the first side wall 111 and form the section where it is connected to the connecting part 122 (i.e., a partial that connects the connecting part 122 to the first side wall 111). The second step is to heat seal the open edges of the first and second side walls 111, 112 to form the main body 11. Herein, the first surface S1 of the fixing parts 12 has the center area thereof connecting to the external surface of the first side wall 111 of the main body 11 thereof via the section where it is connected to the connecting part 122, and a distance D is formed between the edge of the connected area and the edge of the fixing parts 12. The distance D has the maximum of, for example, 3 millimeters, better 8 millimeters, preferably 15 millimeters, to form a detached area 123 of the fixing parts 12. Therefore, there is also a distance between the edge of the connected area and the edge of the main body 11, and, with the distance D, the detached area 123 of the fixing part 12 is detached from the external surface of the main body 11. As the force of pulling is the major cause of the main body 11 detaching and falling from the body surface, including the pulling force from gravity and the one occurs when the user is shifting and causing friction to the main body 11, the detached area 123 of the fixing part 12 being detached from the main body 11 offers the effect of alleviating the pulling forces.

The bag 10 for attaching to body surface of users may further include a release agent cover 15 covering at least parts of the adhesive layer 14, preventing the latter from loss of stickiness due to contamination. The release agent covers 15 can be, for example, release papers. Moreover, referring to FIGS. 3A and 3C, the center area of fixing parts 12 is connected to the main body 11 via the connecting part 122. Herein, the connecting part 122 is in a closed annular shape to connect the first side wall 111 and the first surface S1 of the fixing parts 12. And the connecting part 122 is arranged near the upper edge of the main body 11 and the inner edge of the bonded section 113, rendering the upper end of the detached area 123 of the fixing parts 12 extending out from the edge of the main body 11. That is, the fixing parts 12 can extend toward the edge of the main body 11, so that the edge of the fixing parts 12 protrudes beyond the edge of the main body 11. If here we perform the same manufacturing process as the one of FIG. 3B, the heat-sealing process in the second step would bond the open edges of the first and second side walls 111, 112 to form the main body 11, because they are thermoplastic films heated at the same time (the first side wall 111, the second side wall 112 and the fixing part 12), the edge of the main body 11 (or the first side wall 111) will be combined with the upper end of the detached area 123 of the fixing part 12 at the same time, causing the main body 11 to lose the aforementioned relationship between the fixing part 12 and the separation part between the main body 11 has the effect of buffering the pulling force. In order to avoid the detached area 123 of the fixing part 12 and the edge of the main body 11 being heated and combined with each other, the bag 10 further includes an insulation layer 16 arranged on the surface of the main body. Herein, it is arranged on the inner surface of the main body 11 for exemplary purposes; in other embodiments, the insulation layer 16 can also be arranged on the external surface of the main body 11 or the first surface S1 of the fixing part 12. For instance, with the manufacturing process described above, the first step is preferred to heat seal the open edges of the first and second side walls 111, 112 to form the main body 11. Then the second step is to heat seal the fixing parts 12 with the first side wall 111 and produce the connecting part 122. At this point, to prevent from the inner surface of the second side wall 112 being heat sealed with the first side wall 111, the insulation layer 16 can be arranged on either of the corresponding surfaces of two walls via being disposed of in-between, applying a layer, attaching, or printing before the first and second side walls 111, 112 are bonded to form the main body 11. The techniques applied in the present invention should be comprehendible for those who possess common knowledge. In this embodiment, the insulation layer 16 can also be arranged on the fixing parts 12, for example, on the first surface S1 of the detached area 123. This can also prevent the detached area 123 of the fixing parts 12 from being bonded with the edge of the main body 11 by the heat. Furthermore, the bag 10 can further include a second opening 121 arranged in an area within a circular connecting section of the fixing parts 12, reducing the coverage area of the adhesive layer (not shown) attached to the body surface of the users without compromising the effectiveness of the fixed attachment, thereby increasing the comfort for the skins. Additionally, a strong adhesive layer 141 can be further arranged at one side of the adhesive layer 14 in the central area of the fixing parts 12, allowing the center area of the fixing parts 12 to be more strongly attached to the body surface as well as prolonging the period before the fixing parts 12 become detached from the body surface. Wherein the strong adhesive layer 141 can be formed by spot gluing, applying a layer, or printing; or it can be a double-sided tape. The bag 10 as shown in FIG. 3C has the main body 11 thereof presenting in a droplet shape when being attached to a body surface and carrying weights due to the gravity force (from top to down direction in the figure), as illustrated in FIG. 3D. The upper section of the main body 11, where it is narrower is smoothly attached to the fixing part 12. When comparing with FIG. 3B, it can effectively reduce the risk that the bag 10 fixed on the surface of the body will fall off from the surface of the body due to friction on the top of the main body 11. What's worth mentioning is that, the farther the connecting part 122 of the bag 10 is from the gravity direction, the more effective the attachment is. In FIG. 3D, the main body 11 is attached to the body surface of the user with the adhesive layer 14 of the fixing part 12. The first surface S1 below the fixing part 12 (separation part) is separated from the outer surface of the main body 11 can alleviate the gravity force or the pulling force from friction. Below the first side wall 111, there is another fixing part 12 having both corresponding sides thereof attached to the main body 11 for the fixed member 21 (such as straps and ropes) to pass through the passage. With reference to FIG. 2A, the lower section of the main body 11 can be further fixedly attached to other areas of the body surface. Herein, the main body 11 includes the extended element like a drainage tube in the first opening 13 at the second side wall 112, to drain the substance in the storage space of the main body 11. The techniques applied in the present invention should be comprehendible for those who possess common knowledge. In this embodiment, the extended element in the first opening 13 can be but is not limited to a tube, a connector, a cover, and/or the adhesive layer surrounding the first opening 13.

The present invention has been disclosed in the preferred embodiment above. However, it can be appreciated by one of ordinary skill in the art that these embodiments are illustrative rather than limitations of the scope of the present invention. It should be noted that the above embodiments can be modified and replaced with equivalents thereof without departing from the scope of the present invention. Therefore, the scope claimed of the present invention should only be defined by the following claims.

### REFERENCE SIGNS

10: bag
11: main body
111: first side wall
112: second side wall
113: inner edge of the bonded section
12: fixing part
121: second opening
122: connecting part
123: detached area
13: first opening
14: adhesive layer
141: strong adhesive layer
15: release agent cover
16: insulation layer
21: fixed member
22: reinforcement
S1: first surface
S2: second surface
d: interval
D: distance

What is claimed is:

1. A bag arranged on the body surface, comprising:
a main body including a first side wall, a second side wall, a storage space, and a first opening, said first side wall bonded with said second side wall forming said storage space, said first opening formed at least at said first side wall, at said second side wall or in-between said first and second side walls and said storage space connecting to outside the main body through said first opening;
a plurality of fixing parts individually arranged on the main body, each of fixing parts including a first surface and a second surface which are correspondingly arranged, said second surface being attachable on a body surface and said first surface being attachable on said first side wall; and
a connecting part arranged in-between the main body and the fixing parts, to fix the fixing parts on the main body, wherein said connecting part connects partial of the first surface and the first side wall, rendering the rest of the first surface detached from the first side wall;
wherein the main body is attachable by the plurality of fixing parts as well as the connecting part and thereby fixed on a body surface.

2. The bag defined in claim 1, wherein the plurality of fixing parts includes intervals arranged in-between, said intervals spaced no less than 0 cm and arranged at least in the same direction or in a staggered position.

3. The bag defined in claim 1, further comprises a reinforcement arranged at least on the first surface or the second surface of at least one of the fixing parts.

4. The bag defined in any one of claims 1-3, wherein one of the fixing parts comprises a passage formed by at least two connecting parts for arranging an external fixed member therethrough.

5. The bag defined in any one of claims 1-3, further comprises an extended element, partial of said extended element is exposed outside the main body, and another partial thereof connecting to the first opening.

6. The bag defined in any one of claims 1-3, further comprises a second opening formed at least at one of the fixing parts.

7. A bag arranged on the body surface, comprising:
a main body including a first side wall, a second side wall, a storage space, and a first opening, said first side wall bonded with said second side wall, forming said storage space, said first opening formed at least at said first side wall, at said second side wall or in-between said first and second side walls, and said storage space connecting to outside the main body through said first opening;
a plurality of fixing parts individually arranged on the main body, each of fixing parts including a first surface and a second surface which are correspondingly arranged, said second surface being attachable on a body surface and said first surface being attachable on said first side wall;
a connecting part arranged in-between the main body and the fixing parts, to fix the fixing parts on the main body, wherein said connecting part connects partial of the first surface and the first side wall, rendering the rest of the first surface detached from the first side wall; and
an adhesive layer formed on the second surface;
wherein the main body is attachable with the plurality of fixing parts, the connecting part, and the adhesive layer and thereby fixed on a body surface.

8. The bag defined in claim 7, wherein one of the fixing parts has said partial of the first surface thereof arranged in a distance from which to an edge of the fixing part.

9. The bag defined in claim 7, wherein the connecting part is arranged near an edge of the main body, rendering an edge of at least one of the fixing parts has at least partial thereof extending out from the edge of the main body.

10. The bag defined in any one of claims 7-9, wherein the connecting part is in an annular shape to connect the first side wall and said partial of the first surface of the fixing part.

11. The bag defined in any one of claims 7-9, further comprises an extended element, partial of said extended element exposed outside the main body, and another partial thereof connecting to the first opening.

12. The bag defined in any one of claims 7-9, further comprises a second opening formed at least at one of the fixing parts.

13. The bag defined in any one of claims 7-9, further comprises an insulation layer, arranged at least on an inner surface of the main body, an external surface of the main body, or on the first surface.

14. The bag defined in any one of claims 7-9, further comprises a strong adhesive layer arranged at one side of the adhesive layer.
